(19) United States
(12) Patent Application Publication
(10) Pub. No.: US 2025/0288534 A1
(43) Pub. Date: Sep. 18, 2025

(54) COMPOSITIONS AND METHODS FOR PEPTIDE OR PROTEIN DELIVERY TO THE CENTRAL NERVOUS SYSTEM

(71) Applicants: The University of British Columbia, Vancouver (CA); Nanovation Therapeutics Inc., Vancouver (CA)

(72) Inventors: Louis-Philippe BERNIER, Vancouver (CA); Brian MACVICAR, Vancouver (BC); Pieter R. CULLIS, Vancouver (CA); Jayesh A. KULKARNI, Vancouver (CA)

(21) Appl. No.: 19/226,821

(22) Filed: Jun. 3, 2025

Related U.S. Application Data
(63) Continuation of application No. PCT/CA2023/051628, filed on Dec. 7, 2023.
(60) Provisional application No. 63/430,740, filed on Dec. 7, 2022.

(51) Int. Cl. A61K 9/51; A61K 31/7105; A61K 47/28; A61P 25/00

(52) U.S. Cl. CPC A61K 9/5146; A61K 9/5123; A61K 31/7105; A61K 47/28; A61P 25/00

(57) ABSTRACT

The present disclosure provides a lipid nanoparticle to treat, prevent or diagnose a central nervous system disease, disorder, trauma or injury, the lipid nanoparticle comprising: a non-cationic helper lipid; a sterol; a hydrophilic polymer-lipid conjugate; an ionizable, amino amino lipid having a pKa between 5.0 and 7.0; and an mRNA having a nucleic acid sequence encoding for a secretory polypeptide for treating, preventing or diagnosing the central nervous system disease, disorder, trauma or injury, the secretory polypeptide being capable of secretion from a cell of the central nervous system into a interstitial and/or cerebrospinal fluid of a subject. Further provided are methods for administration of the lipid nanoparticles to treat, prevent or diagnose the central nervous system disease, disorder, trauma or injury and uses of such lipid nanoparticles.

### CROSS REFERENCE TO RELATED APPLICATIONS

This application is a continuation of International Patent Application No. PCT/CA2023/051628, filed 7 Dec. 2023, which claims the benefit of priority to U.S. Provisional Patent Application Ser. No. 63/430,740, filed 7 Dec. 2022. The disclosure of the above-referenced application is herein expressly incorporated by reference in its entirety, including any drawings.

### TECHNICAL FIELD

The present disclosure relates to compositions and methods for treating, preventing or diagnosing a central nervous system diseases or disorders. In particular, the compositions include lipid nanoparticles (LNPs) for the delivery of mRNA (LNP-mRNA).

### BACKGROUND

Neurological disorders are often chronic conditions that bring a heavy burden to patients, their families and the health care system [4]. Extensive investigations on potential treatments have been ongoing for decades, but the clinical success rate of therapy targeted to the central nervous system (CNS) is low when compared to other organs [6]. It is widely believed that beyond the complex nature of neural communication, a major hurdle for brain therapy is delivery of a therapeutic [1] to the target site or sites. The brain is particularly difficult to target, largely due to the presence of the blood-brain barrier (BBB), shielding the brain parenchyma from the periphery and preventing the passage of most small molecules and nearly all macromolecules [7, and 11].

Although the translation of research into clinical applications has been slow, there has been progress in understanding the molecular basis of many neurologic disorders and diseases. For example, the pathophysiology of highly prevalent diseases such as stroke, traumatic brain injury (TBI), neurodegenerative diseases and glioblastoma are now well understood, and several specific signaling pathways are known to hold therapeutic promise. A wealth of knowledge exists describing how changes in expression of specific proteins affect, or sometimes directly drive, disease progression and maintenance. In virtually all CNS diseases, the absence or downregulation of one or many endogenous proteins is known to participate in the pathology of the disease. Accordingly, making protein replacement or supplementation an appealing therapeutic modality [3]. However, these disease mechanisms have remained difficult to manipulate pharmacologically.

Despite advances in the synthesis of recombinant proteins, several drawbacks to direct supplementation reduce their potential in CNS therapeutic applications. The main challenges remain the inability of proteins to penetrate the BBB and access the brain parenchyma, as well as the short half-life of proteins, which restricts the time course of therapeutic effects. An LNP strategy has been used to introduce small interfering RNAs (siRNA) into neurons in vivo to disrupt expression of SLC26A11 determined to be critical and required for neuronal swelling [12, and 13]. Similarly, mRNA encapsulated in SS-cleavable proton-activated lipid-like material (ssPalm) nanoparticles was shown to deliver exogenous mRNA, encoding proteins, into neuronal cells and astrocytes via intracerebroventricular (ICV) administration [14]. Intrathecal lumbar injection of LNP-mRNA was found to mostly target the dorsal root ganglia [9], measured by intracellular protein production. Similarly, transfecting neurons, astrocytes, and microglia/macrophages with and LNP-mRNA construct encoding human IL-10 in the area of a spinal cord lesion promoted neuroprotection and functional recovery in a spinal cord injury model[5, and 10]. Accordingly, them appears to be a therapeutic potential for LNP-mRNA as an alternative to previous approaches to treat, prevent or diagnose a central nervous system disease, disorder, trauma or injury.

### SUMMARY

The present disclosure seeks to address one or more problems in the state of the art to treat, prevent or diagnose a central nervous system disease, disorder, trauma or injury.

This invention is based in part on the fortuitous discovery that LNP-mRNA formulations can deliver mRNA to cells of the brain (e.g., glial cells) when injected in the brain via intracerebroventricular (ICV) injection or lumbar intrathecal injection. Furthermore, particular LNP-mRNA compositions when injected into the cerebrospinal fluid (CSF) preferentially target glial cells to express the peptide or protein (herein “polypeptide”) encoded by the mRNA and then subsequently to secrete therapeutically effective amounts of peptide or protein into the CSF. In some embodiments, the LNP-encapsulated mRNA causes glial cells in the brain to produce and secrete a desired peptide or protein into the extracellular milieu of the brain parenchyma. Specifically, the therapeutically effective amount of secreted peptides or proteins may act extracellularly or be taken up by target cells. Alternatively, such proteins or peptides may have diagnostic value within the CSF. In particular, the glial cells are oligodendrocytes or astrocytes. The desired peptide or protein encoded by the mRNA thereby can achieve therapeutically effective amounts in the CSF when measured distally from the injection site.

The approach presented here delivers genetic material (mRNA) via direct injection (e.g., intracerebroventricular or intrathecal injection) to a subset of brain cells to instruct these cells to translate the mRNA into a desired protein, which is then secreted to the extracellular space. The desired protein then diffuses throughout the brain compartment and can be measured at significant concentrations within the cerebrospinal fluid (CSF) collected distally from the initial injection site. Importantly, these cells continue translating the mRNA into protein for an extended period of time related to the mRNA half-life, thereby achieving persistent delivery of protein to the brain extracellular milieu and CSF.

According to one aspect of the disclosure, there is provided a method for treating, preventing or diagnosing a central nervous system disease, disorder, trauma or injury, the method comprising: contacting a lipid nanoparticle (LNP) with a cell of the central nervous system of a subject, the lipid nanoparticle comprising:
(a) an ionizable, amino, cationic lipid having a pKa between 5.0 and 7.0;
(b) a non-cationic helper lipid;
(c) a sterol;
(d) a hydrophilic polymer-lipid conjugate; and
(e) an mRNA encoding for a secretory polypeptide for treating, preventing or diagnosing the central nervous system disease, disorder, trauma or injury, the secretory polypeptide being capable of secretion from a cell of the central nervous system into a interstitial and/or cerebrospinal fluid of the subject, wherein the contacting of the LNP with the cell results in the mRNA entering the cell and being translated in the cell, thereby resulting in production of the polypeptide,
   wherein, subsequent to the production of the polypeptide, the polypeptide is secreted from the cell to an interstitial and/or cerebrospinal fluid (CSF) of the subject, wherein the secreted polypeptide is present in the CSF of the subject at a first concentration at a first time point and at a second concentration at a later second time point, wherein the first time point is 3 hours after contacting the LNP with the cell and the second time point is 48 hours after contacting of the LNP with the cell, wherein the second concentration is at least 50%, at least 60%, at least 70%, at least 80%, at least 90%, or at least 95% of the first concentration, and wherein the contacting is in vitro or in vivo.

In one embodiment, the cell of the central nervous system is selected from one or more of: (a)
a glial cell; and (b) an ependymal cell.

In another embodiment, the glial cell is selected from an astrocyte or oligodendrocyte and the ependymal cell is from the cerebral ventricles or the choroid plexus.

According to a further embodiment, the secretory polypeptide is endogenous to the cell of the central nervous system or modified to enhance its activity in the brain.

According to another embodiment, the secretory polypeptide is diffusible within the interstitial and/or cerebrospinal fluid of the subject.

According to another embodiment, the non-cationic helper lipid is a generally cylindrically-shaped lipid.

In another embodiment, the non-cationic helper lipid is distearoylphosphatidylcholine (DSPC) or dioleoylphosphatidylglycerol (DOPG).

In another embodiment, the non-cationic helper lipid has a phosphatidylethanolamine content that is less than 2 mol %.

According to a further embodiment, the sterol is cholesterol.

In a further embodiment, the polymer lipid conjugate is PEG-DMG.

In another embodiment, the molar ratio of ionizable, amino, cationic lipid/non-cationic helper lipid/sterol/polymer lipid conjugate is 50/10/38-39/1-2.

According to a further embodiment, the method comprises injection via an intracerebroventricular (ICV) or lumbar intrathecal route, cisterna magna route or via a catheter to the central nervous system of the subject.

According to a further aspect, there is provided a use of a pharmaceutical formulation comprising a lipid nanoparticle (LNP) for contacting a cell of the central nervous system of a subject to treat, prevent or diagnose a central nervous system disease, disorder, trauma or injury, the lipid nanoparticle comprising:
(a) an ionizable, amino, cationic lipid having a pKa between 5.0 and 7.0;
(b) a non-cationic helper lipid;
(c) a sterol;
(d) a hydrophilic polymer-lipid conjugate; and
(e) an mRNA encoding for a secretory polypeptide to treat, prevent or diagnose the central nervous system disease, disorder, trauma or injury, the secretory polypeptide being capable of secretion from a cell of the central nervous system into a interstitial and/or cerebrospinal fluid of a subject after administration of the lipid nanoparticle,
   wherein the contacting of the LNP with the cell results in the mRNA entering the cell and being translated in the cell, thereby resulting in production of the polypeptide,
   wherein, subsequent to the production of the polypeptide, the polypeptide is secreted from the cell to a interstitial and/or cerebrospinal fluid (CSF) of the subject,
   wherein the secreted polypeptide is present in the CSF of the subject at a first concentration at a first time point and at a second concentration at a later second time point,
   wherein the first time point is 3 hours after contacting the LNP with the cell and the second time point is 48 hours after contacting of the LNP with the cell,
   wherein the second concentration is at least 50%, at least 60%, at least 70%, at least 80%, at least 90%, or at least 95% of the first concentration, and
   wherein the contacting is in vitro or in vivo.

According to another aspect, there is provided a use of a lipid nanoparticle for contacting a cell of the central nervous system of a subject for the manufacture of a medicament to treat, prevent or diagnose a central nervous system disease, disorder, trauma or injury, the lipid nanoparticle comprising:
(a) an ionizable, amino, cationic lipid having a pKa between 5.0 and 7.0;
(b) a non-cationic helper lipid;
(c) a sterol;
(d) a hydrophilic polymer-lipid conjugate; and
(e) an mRNA having a nucleic acid sequence encoding for a secretory polypeptide
   to treat, prevent or diagnose the central nervous system disease, disorder, trauma or injury, the secretory polypeptide being capable of secretion from a cell of the central nervous system into a interstitial and/or cerebrospinal fluid of a subject after administration of the lipid nanoparticle, wherein the contacting of the LNP with the cell results in the mRNA entering the cell and being translated in the cell, thereby resulting in production of the polypeptide, wherein, subsequent to the production of the polypeptide, the polypeptide is secreted from the cell to a interstitial and/or cerebrospinal fluid (CSF) of the subject, wherein the secreted polypeptide is present in the CSF of the subject at a first concentration at a first time point and at a second concentration at a later second time point, wherein the first time point is 3 hours after contacting the LNP with the cell and the second time point is 48 hours after contacting of the LNP with the cell, wherein the second concentration is at least 50%, at least 60%, at least 70%, at least 80%, at least 90%, or at least 95% of the first concentration, and wherein the contacting is in vitro or in vivo.

In another embodiment, the cell of the central nervous system is a glial cell selected from an astrocyte or oligodendrocyte or an ependymal cell of the cerebral ventricles or the choroid plexus.

In a further embodiment of the foregoing use, the non-cationic lipid is a generally cylindrically-shaped lipid.

In another embodiment of the foregoing use, the non-cationic lipid is distearoylphosphatidylcholine (DSPC) or dioleoylphosphatidylglycerol (DOPG).

In another embodiment of the foregoing use, the phosphatidylethanolamine content is less than 2 mol %.

In another embodiment of the foregoing use, the sterol is cholesterol.

In a further embodiment of the foregoing use, the polymer lipid conjugate is PEG-DMG.

In another embodiment of the foregoing use, the molar ratio of ionizable, amino, cationic lipid/non-cationic helper lipid/sterol/polymer lipid conjugate is 50/10/38-39/1-2.

In another embodiment of the foregoing use, the therapeutic polypeptide or peptide is endogenous to the cell of the central nervous system or modified to enhance its activity in the brain.

In another embodiment of the foregoing use, the therapeutic polypeptide or peptide is diffusible within the interstitial and/or cerebrospinal fluid.

According to another aspect of the disclosure, there is provided a lipid nanoparticle for contacting a cell of the central nervous system of a subject to treat, prevent or diagnose a central nervous system disease, disorder, trauma or injury, the lipid nanoparticle comprising:
(a) an ionizable, amino, cationic lipid having a pKa between 5.0 and 7.0;
(b) a non-cationic helper lipid;
(c) a sterol;
(d) a hydrophilic polymer-lipid conjugate; and
(e) an mRNA having a nucleic acid sequence encoding for a secretory polypeptide for treating, preventing or diagnosing the central nervous system disease, disorder, trauma or injury, the secretory polypeptide being capable of secretion from a cell of the central nervous system into a interstitial and/or cerebrospinal fluid of a subject,
   wherein the contacting of the LNP with the cell results in the mRNA entering the cell and being translated in the cell, thereby resulting in production of the polypeptide,
   wherein, subsequent to the production of the polypeptide, the polypeptide is secreted from the cell to a interstitial and/or cerebrospinal fluid (CSF) of the subject,
   wherein the secreted polypeptide is present in the CSF of the subject at a first concentration at a first time point and at a second concentration at a later second time point,
   wherein the first time point is 3 hours after contacting the LNP with the cell and the second time point is 48 hours after contacting of the LNP with the cell,
   wherein the second concentration is at least 50%, at least 60%, at least 70%, at least 80%, at least 90%, or at least 95% of the first concentration, and
   wherein the contacting is in vitro or in vivo.

According to another aspect of the disclosure, there is provided a method for treating, preventing or diagnosing a central nervous system disease, disorder, trauma or injury the method comprising: administering a lipid nanoparticle to the central nervous system of a subject, the lipid nanoparticle comprising: a neutral lipid; a sterol; a hydrophilic polymer-lipid conjugate; an ionizable, amino lipid having a pKa below 7.0; and an mRNA encoding for a secretory protein for treating, preventing or diagnosing the central nervous system disease, disorder, trauma or injury, the secretory protein being capable of secretion from a cell of the central nervous system into a cerebrospinal fluid of the subject.

In one embodiment of the foregoing aspect, the cell of the central nervous system is a glial cell selected from an astrocyte or oligodendrocyte or an ependymal cell of the cerebral ventricles or the choroid plexus.

In another embodiment of the foregoing aspect, the lipid nanoparticle is part of a pharmaceutical formulation.

In another embodiment of the foregoing aspect, the secretory polypeptide is endogenous to the cell of the central nervous system or modified to enhance its activity in the brain.

In a further embodiment of the foregoing aspect, the secretory polypeptide is diffusible within the interstitial and/or cerebrospinal fluid of the subject.

In a further embodiment of the foregoing aspect, the non-cationic helper lipid is a generally cylindrical-shaped lipid.

According to a further embodiment of the foregoing aspect, the non-cationic lipid is distearoylphosphatidylcholine (DSPC) or dioleoylphosphatidylglycerol (DOPG).

According to a further embodiment of the foregoing aspect, the non-cationic helper lipid has a phosphatidylethanolamine content that is less than 2 mol %.

According to a further embodiment of the foregoing aspect, the sterol is cholesterol.

According to a further embodiment of the foregoing aspect, the polymer lipid conjugate is PEG-DMG.

According to a further embodiment of the foregoing aspect, the molar ratio of ionizable, amino, cationic lipid/non-cationic helper lipid/sterol/polymer lipid conjugate is 50/10/38-39/1-2.

Alternatively, the ionizable, amino, cationic lipid having a pKa between 5.0 and 7.0 may be nor-MC3 and or compound 22.

In a further example of any of the foregoing aspects or embodiments of the disclosure, the administering may comprise injection via an intracerebroventricular (ICV), lumbar intrathecal route, cisterna magna route or via a catheter to the central nervous system of the subject.

In a further example of any of the foregoing aspects or embodiments of the disclosure, the lipid nanoparticle may be part of a pharmaceutical formulation that is injectable via an intracerebroventricular (ICV) route, lumbar intrathecal route, cisterna magna route or via a catheter to the central nervous system of the subject.

### BRIEF DESCRIPTION OF THE FIGURES

FIG. 1 depicts a proposed mechanism for delivery of a secretory protein to the central nervous system using an LNP-mRNA as described herein.

FIG. 2 shows a schematic of an intracerebroventricular (ICV) injection of LNP-mRNA to the brain of a mouse, where the LNP-mRNA encodes for the fluorescent reporter protein mCherry™, bilaterally injected in mice (lower right). The images at the left show mCherry™ expression in brain slices of the white matter tract of the corpus callosum at 2 days post-injection, where mCherry™ was used as a reporter to visualize cells translating the mRNA cargo into mCherry™ protein, but mCherry™ is not secreted (arrow indicates the direction of slices taken from the posterior to the anterior of the corpus callosum). The cells instructed to express mCherry™ have a widespread distribution in the brain, but most pronounced mCherry™ protein is found in the corpus callosum.

FIG. 3A shows brain images of LNP-mRNA uptake in oligodendrocytes (olig2+), following ICV injection, uptake of LNP-mRNA encoding for mCherry™ induced expression of mCherry™ that was visualized within immunostained oligodendrocytes in white matter tracts in mice. The broken lined box in the upper images (100 μm scale) are the areas enlarged in the corresponding lower images (20 μm scale).

FIG. 3B shows brain images of LNP-mRNA uptake in astrocytes (GFAP+), following ICV injection, uptake of LNP-mRNA encoding for mCherry™ induced expression of mCherry™ that was visualized within immunostained astrocytes in white matter tracts in mice. The broken lined box in the upper images (100 μm scale) are the areas enlarged in the corresponding lower images (20 μm scale).

FIG. 4 shows brain images following intracerebroventricular (ICV) injection of the LNP-mRNA encoding for mCherry™ in mice. The images show immunostaining of cells expressing mCherry™ in the choroid plexus and ventricle walls in the mice at various magnifications.

FIG. 5 shows spinal cord images of LNP-mRNA uptake. LNPs with encapsulated mRNAs encoding for mCherry™ were injected via the intrathecal lumbar route in a mouse, and the animal was perfused and fixed tissue was collected two days after LNP-mRNA injection. The images show immunostaining of cells in the spinal cord expressing mCherry™.

FIG. 6 shows a schematic of the protocol used to demonstrate that LNP-mRNA can induce production and secretion of a desired protein to the CSF. The concentration of FGF21 protein in cerebrospinal fluid (CSF) was measured after bilateral ICV injection in mice of two different formulations of LNP-mRNA (i.e. LNP(1)-FGF-21 (LNP-G FGF-21) and LNP(2)-FGF-21 (LNP-H FGF-21)) coding for FGF-21 (graph at the right side of the figure) as compared to CSF control and LNP-mRNA EPO negative control (i.e. mRNA encoding EPO does not yield increases in FGF-21). This demonstrates the specificity of the assay but importantly that protein production and secretion is specific to mRNA encoded protein. After LNP-mRNA FGF-21 injection CSF was collected and FGF-21 was measured using a U-PLEX FGF21 assay.

FIG. 7 shows changes in CSF FGF-21 concentrations at 3 hours and 48 hours post injection in response to varying doses of LNP-mRNA encoding for FGF-21 (LNP(2)-FGF-21) injected ICV. CSF FGF-21 protein was measured using a quantitative U-PLEX assay. Dashed line corresponds to CSF FGF-21 concentration at 3 hours post-injection at a dose of 1 mg/mL mRNA.

FIG. 8 shows the concentration of EPO protein in CSF after ICV injection of LNP-mRNA encoding the secreted factor EPO (LNP-EPO) in mice (graph at the right side of the figure), where after injection, CSF was collected and EPO was measured using a U-PLEX EPO assay as described herein (depicted in the left side of the figure). Control CSF is a negative control from un-injected animals, and LNP-FGF-21 is a negative injection control showing LNP-mRNA FGF-21 production does not alter CSF EPO concentrations.

FIG. 9 shows changes in CSF EPO concentrations at 3 hours and 48 hours post injection in response to varying doses of LNP-mRNA encoding for EPO (LNP-EPO) injected ICV. CSF EPO protein was measured using a quantitative U-PLEX assay. Dashed line corresponds to CSF [EPO]3 hours post-injection at a dose of 1 mg/mL mRNA.

FIG. 10 shows coronal and sagittal views of the mouse brain with the intracerebroventricular (ICV) injection site (grey triangle “ICV inject”—coronal and sagittal), with the lateral ventricle (LV) injections sites in black and cerebral spinal fluid (CSF) collections site (grey triangle “CSF collect”—sagittal only) and the approximate distance between the injection and collection sites (i.e. 8 mm—sagittal only).

### DETAILED DESCRIPTION

The following detailed description will be better understood when read in conjunction with the appended figures. For the purpose of illustrating the invention, the figures demonstrate embodiments of the present invention. However, the invention is not limited to the precise arrangements, examples, and instrumentalities shown.

The present disclosure provides compositions for treating, preventing or diagnosing a central nervous system condition, disease, disorder, trauma or injury using lipid nanoparticles comprising mRNA encoding a secretory protein, also referred to herein as “LNP-mRNA”. Delivery of the LNP-mRNA to a glial cell may convert the glial cell into a “CNS protein bioreactor” that secretes the protein or peptide encoded by the mRNA. Without being bound by any particular theory, an example of the inventive delivery method is shown in FIG. 1. With reference to the example depicted in FIG. 1, a lipid nanoparticle comprising an mRNA that encodes a secretable therapeutic protein is injected into the CNS of a subject (i.e. intracerebroventricular (ICV) injection; lumbar intrathecal injection (IT); or cisterna magna injection), which is depicted as a mouse by way of example, but includes any mammalian subject, including a human. The LNP-mRNA may be taken up by endocytosis into brain cells (e.g., glial cell) and the mRNA is translated into the protein in the cytoplasm of the cell. The secretory protein is subsequently secreted from the cell into an interstitial and/or cerebrospinal fluid (CSF), allowing the protein to diffuse to a target site and exert a therapeutic, diagnostic or prophylactic effect in one or more regions of the central nervous system. In alternative embodiments, the protein is a diagnostic agent. As depicted, in some advantageous examples of the disclosure, the protein secreted by the glial cell is diffusible, thereby effecting brain-wide delivery within a subject. A variety of secretory proteins, peptides, combinations of two or more proteins, combinations of two or more peptides or combinations of proteins and peptides can be delivered to the central nervous system. The mRNA may encode proteins or peptides, including but not limited to antibodies, growth factors and other therapeutic, diagnostic or prophylactic proteins described herein.

The lipid nanoparticle in some embodiments comprises four lipid components, as described herein. In some embodiments, this includes an ionizable lipid, a helper lipid, a hydrophilic polymer-lipid conjugate (e.g., a PEG-lipid) and cholesterol or other sterol as described herein. Such LNP compositions are shown herein to be particularly efficacious in the delivery of mRNA to a cytoplasm of a cell of the central nervous system where it is translated into a secretory protein that is ultimately secreted into the interstitial and/or cerebrospinal fluid.

Previous studies have demonstrated that LNP-mRNA uptake in neural cells in the CNS is possible with direct ICV or IT injection. These studies use LNP-mRNA to generate intracellular proteins either as reporter markers to confirm LNP-uptake [14], or to express a biologically active protein [6, and 14]. Precedent work relies on direct LNP-uptake in diseased/dysregulated cell types to treat a neurological disorder by altering the local microenvironment via mRNA-produced protein. The present disclosure instead uses a consistent subpopulation of neural cells, namely glial cells, as CNS protein bioreactors specifically to produce and secrete proteins for brain-wide bio-distribution and at therapeutic concentrations.

### Secretory Protein

The protein or peptide encoded by the mRNA of the lipid nanoparticle is collectively referred to herein as a “secretory protein”, meaning that the protein can cross a central nervous system cellular membrane to exert an extracellular therapeutic or prophylactic effect within one or more regions of the central nervous system. The secretory protein may comprise a secretion signal that facilitates secretion thereof. The protein may already possess a secretion signal sequence (e.g., growth factors and cytokines) or may be genetically modified to include a secretion signal sequence. Without being limiting, the secretion signal may be located at the amino terminus of the secretory protein and cause the translocation of newly synthesized protein through the endoplasmic reticulum, Golgi network and to the cell membrane for secretion into interstitial and/or cerebrospinal fluid. Alternatively, in one example of the disclosure, the secretory protein is secreted via a “non-classical” pathway that is not reliant on the inclusion of a secretion signal. Moreover, secretory proteins may be naturally released by central nervous system cells, such as astrocyte or oligodendrocyte or an ependymal cell of the cerebral ventricles or the choroid plexus under specific environmental conditions.

The ability of a protein or peptide to be secreted from a central nervous system cell can be assessed in vitro by the method described in EXAMPLE 3 herein. Briefly, the assay involves culturing brain glial cells (astrocytes and oligodendrocytes) and treating the cell cultures with LNP-mRNA coding for the candidate protein. Secretion of the protein is then assayed by collecting the cell supernatant and quantifying the protein amount, for example, via immunoblotting, ELISA or a U-PLEX assay. The supernatant will be collected at various time points to estimate the peak production/secretion timeline. The in vitro assay will typically be followed up with in vivo studies in rodents to confirm protein secretion. Such in vivo studies are described in the Examples section herein.

For some proteins where intracellular localization after secretion is desirable, a sequence coding for a cell-penetrating peptide (CPP) such as TAT peptide may be included. Genetically engineered mRNA sequences could therefore include both a signal sequence and a CPP sequence to induce secretion and promote cellular uptake of the protein by a target cell, respectively.

The secretory protein may treat or prevent a central nervous system disease, disorder, trauma or injury. Typically, the protein is secreted from the cell of the central nervous system into the cerebrospinal fluid of the subject and diffuses to a target site.

The LNP-mRNAs encoding the secretory protein may also be used in other applications besides the treatment and/or prevention of a disease, disorder, trauma or injury. The LNP-mRNAs may be used to treat conditions such as aging, preventative medicine and/or as part of a personalized medicine regime. In further embodiments, the LNP may be used in a diagnostic application.

Non-limiting examples of diseases, disorders, traumas or injuries and secretory proteins that may be used to treat them are provided in TABLE 1 below.

TABLE 1
Central nervous system diseases or disorders and secretory
proteins to treat such diseases or disorders

| Disease/Disorder | Secretory protein |
|---|---|
| Stroke | Brain-derived neurotrophic factor (BDNF) |
| | Vascular endothelial growth factor (VEGF) |
| | Nerve growth factor (NGF) |
| | Neurotrophin-3 (NT-3) |
| | Granulocyte colony stimulating factor (G-CSF) |
| | Granulocyte macrophage colony stimulating factor (GM- CSF) |
| | Stem cell factor (SCF) |
| | Stromal cell-derived factor 1α (SDF-1α) |
| | Erythropoietin (EPO) |
| | Insulin growth factor-1 (IGF-1) |
| | Fibroblast growth factors (FGF1, FGF2, FGF10, FGF21) |
| Traumatic brain injury (TBI) | Angiopoietin-1 (Ang-1) |
| | Granulocyte colony stimulating factor (G-CSF) |
| | Stem cell factor (SCF) |
| | Vascular endothelial growth inhibitor (VEGI) (*VEGI/VEGF |
| | benefits are time-dependent after TBI) |
| | Insulin growth factor-1 (IGF-1) |
| | Nerve growth factor (NGF) |
| | Epidermal growth factor (EGF) |
| | Fibroblast growth factor-2 (FGF2) |
| Alzheimer's disease | Brain-derived neurotrophic factor (BDNF) |
| | Progranulin |
| | Nerve growth factor (NGF) |
| | Interleukin-4 (IL-4) |
| | Interleukin-10 (IL-10) |
| | Tumor growth factor β (TGFβ) |
| Parkinson's disease | Brain-derived neurotrophic factor (BDNF) |
| | Glial cell line-derived neurotrophic factor (GDNF) |
| Multiple sclerosis | Fibroblast growth factor-2 (FGF2) |
| | Insulin growth factor-1 (IGF-1) |
| | Platelet-derived growth factor (PDGF) |
| | Ciliary neurotrophic factor (CNTF) |
| | Brain-derived neurotrophic factor (BDNF) |
| Neuroinflammation | Interleukin-4 (IL-4) |
| | Interleukin-10 (IL-10) |
| | Tumor growth factor β (TGFβ) |
| Frontotemporal dementia | Progranulin |
| (FTD) | |
| Lysosomal storage diseases | Enzyme replacement therapy - different enzyme for each LSD |
| (LSDs) | |
| Spinal cord injury | Fibroblast growth factors (FGF1, FGF2, FGF21) |
| | Nerve growth factor (NGF) |
| | Brain-derived neurotrophic factor (BDNF) |
| | Neurotrophin-3 (NT-3) |
| | Ciliary neurotrophic factor (CNTF) |
| | Glial cell line-derived neurotrophic factor (GDNF) |
| | Insulin growth factor-1 (IGF-1) |
| Type 2 Diabetes | Fibroblast growth factor-1 (FGF1) |
| Glioblastoma | Interferons (IFNs) |
| | TNF-related apoptosis inducing ligand (TRAIL) |
| Other | Klotho (anti-aging, neurodegenerative diseases) |

It should be appreciated that a combination of two or more secretory proteins may be used to treat or prevent a central nervous system disease, disorder, trauma or injury. Alternatively, or in addition, one or more mutations can be introduced to a secretory protein to increase its half-life in the central nervous system. Further, certain secretory proteins may be used to treat or prevent more than one disease indication.

The protein may also be a diagnostic agent to assess patients for a central nervous system disease, disorder, trauma or injury. In another embodiment, the protein may be used prophylactically to prevent a central nervous system disease, disorder, trauma or injury.

Furthermore, the secretory protein may be a protein fragment (i.e. peptide), protein domain or peptide sequence. The protein may also be modified post-translation.

mRNA

As used herein, the term “messenger RNA” or “mRNA”, refers to a polynucleotide that encodes and expresses the secretory protein.

As used herein, the term “encapsulation”, with reference to incorporating the mRNA within a lipid nanoparticle refers to any association of the mRNA with any lipid component or compartment of the lipid nanoparticle. In one example of the disclosure, the mRNA is present in the core of the LNP.

The mRNA as used herein encompasses both modified and unmodified mRNA. In one embodiment, the mRNA comprises one or more coding and non-coding regions. The mRNA can be purified from natural sources, produced using recombinant expression systems and optionally purified, or may be chemically synthesized. Modifications to the mRNA can improve immunogenicity, stability, and translational efficiency and fidelity of the mRNA and thus increase the amount of protein produced from the mRNA.

In those embodiments in which an mRNA is chemically synthesized, the mRNA can comprise nucleoside analogs such as analogs having chemically modified bases or sugars, and/or backbone modifications. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2 thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The mRNAs of the disclosure may be synthesized according to any of a variety of known methods. For example, mRNAs in certain embodiments may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor.

In some embodiments, in vitro synthesized mRNA may be purified before encapsulation in an LNP to remove undesirable impurities including various enzymes and other reagents used during mRNA synthesis.

The present disclosure may be used to formulate and encapsulate mRNAs of a variety of lengths. In some embodiments, the present disclosure may be used to formulate and encapsulate in vitro synthesized mRNA ranging from about 0.1-20 kb, about 1-20 kb, about 1-15 kb, about 1-10 kb, about 5-20 kb, about 5-15 kb, about 5-12 kb, about 5-10 kb, about 8-20 kb, or about 8-15 kb in length. Alternatively, the peptide or protein produced from the mRNA may be between about 500 Da and about 200 kDa Typically, mRNA synthesis includes the addition of a “cap” on the 5′ end, and a “tail” on the 3′ end. The presence of the cap is important in providing resistance to nucleases found in most eukaryotic cells. The presence of a “tail” serves to protect the mRNA from exonuclease degradation.

In some embodiments, mRNAs include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

While mRNA provided from in vitro transcription reactions may be desirable in certain embodiments, other sources of mRNA are contemplated, such as mRNA produced from bacteria, fungi, plants, and/or animals.

### Lipid Nanoparticle Formulation

The lipid nanoparticle (LNP) described herein causes uptake of the messenger RNA into the cytoplasm of a cell of the central nervous system. Such uptake is facilitated by the inclusion of an ionizable lipid. In addition, the LNPs comprise a non-cationic helper lipid component, a sterol and a hydrophilic polymer-lipid conjugate.

### Ionizable Lipid

An ionizable lipid as described herein may be an ionizable, cationic, amino lipid. The ionizable, cationic, amino lipid may have a pKa such that it is positively charged at low pH and near neutral at physiological pH. This allows for electrostatic interactions between the lipid and the negatively charged mRNA during initial formulation. Since the ionizable, cationic, amino lipid is near neutral at physiological pH, toxicity is reduced. Without being limited by theory, after cellular uptake by endocytosis, the acidic environment of the endosome leads to an increase in the net positive charge of the ionizable, cationic. amino lipids, which promotes fusion with the anionic lipids of the endosomal membrane and subsequent membrane destabilization and release of the mRNA into the cytoplasm of the CNS cell for translation into the secretory protein. As noted previously, the translated protein is subsequently routed to the cell membrane via the endoplasmic reticulum and Golgi network for secretion into interstitial and/or cerebrospinal fluid. Alternatively, the ionizable, cationic, amino lipid may be an ionizable, amino cationic lipids as described in WO 2022/246571 [2]. Alternatively, the ionizable, cationic, amino lipid may have a pKa below 7.0.

The neutral form of the ionizable, amino, cationic lipid has a calculated logarithm of the partition coefficient between water and 1-octanol (i.e., a c Log P) greater than 8. In some embodiments, the ionizable, cationic, amino lipid has a pKa that is between 5.0 and 7.0, or more typically between 6.0 and 6.8.

### Non-Cationic Helper Lipid

As used herein, “non-cationic helper lipid” is a neutral or an anionic structural lipid that is capable of formulation in a lipid nanoparticle.

As used herein, “anionic lipid” is a lipid that is negatively charged at physiological pH. A non-limiting example is a phosphatidylglycerol lipid such as 1,2-dioleoyl-sn-glycero-3-phosphoglycerol (DOPG).

As used herein, “neutral lipid” is a structural lipid that is neutral (including net neutral) at physiological pH and that typically includes a lipid selected from sphingomyelin, a phosphatidylcholine lipid or mixtures thereof. The term “neutral lipid” includes zwitterionic lipids.

In some embodiments, the neutral lipid is selected from sphingomyelin, distearoylphosphatidylcholine (DSPC), dioleoylphosphatidylcholine (DOPC), 1-palmitoyl-2-oleoyl-phosphatidylcholine (POPC) and dipalmitoyl-phosphatidylcholine (DPPC). In certain embodiments, the neutral lipid is DOPC, DSPC or sphingomyelin. In one embodiment, the neutral lipid is DOPC. The neutral lipid content may include mixtures of two or more types of different neutral lipids. Alternatively, a “neutral lipid” may be a cylindrically-shaped lipid. A “neutral lipid” may be distearoylphosphatidylcholine (DSPC). A “neutral lipid” may have a phosphatidylethanolamine content that is less than 2 mol %. A “neutral lipid” may be sphingomyelin, a phosphatidylcholine lipid or mixtures thereof. A “neutral lipid” may be selected from sphingomyelin, distearoylphosphatidylcholine (DSPC), dioleoylphosphatidylcholine (DOPC), 1-palmitoyl-2-oleoyl-phosphatidylcholine (POPC) and dipalmitoyl-phosphatidylcholine (DPPC); or mixtures of two or more types of different neutral lipids.

### Sterol

The LNP further includes a sterol in some embodiments. The term “sterol” refers to a naturally-occurring or synthetic compound having a gonane skeleton and that has a hydroxyl moiety attached to one of its rings, typically the A-ring.

Examples of sterols include cholesterol, or a cholesterol derivative, the latter referring to a cholesterol molecule having a gonane structure and one or more additional functional groups.

The cholesterol derivative may be selected from one or more of the following: 0-sitosterol, 3-sitosterol, campesterol, stigmasterol, fucosterol, or stigmastanol, dihydrocholesterol, ent-cholesterol, epi-cholesterol, desmosterol, cholestanol, cholestanone, cholestenone, cholesteryl-2′-hydroxyethyl ether, cholesteryl-4′-hydroxybutyl ether, 30[N—(N′N′-dimethylaminoethyl)carbamoyl cholesterol (DC-Chol), 24(S)-hydroxycholesterol, 25-hydroxycholesterol, 25(R)-27-hydroxycholesterol, 22-oxacholesterol, 23-oxacholesterol, 24-oxacholesterol, cycloartenol, 22-ketosterol, 20-hydroxysterol, 7-hydroxycholesterol, 19-hydroxycholesterol, 22-hydroxycholesterol, 25-hydroxycholesterol, 7-dehydrocholesterol, 5α-cholest-7-en-30-ol, 3,6,9-trioxaoctan-1-ol-cholesteryl-3e-ol, dehydroergosterol, dehydroepiandrosterone, lanosterol, dihydrolanosterol, lanostenol, lumisterol, sitocalciferol, calcipotriol, coprostanol, cholecalciferol, lupeol, ergocalciferol, 22-dihydroegocalciferol, ergosterol, brassicasterol, tomatidine, tomatine, ursolic acid, cholic acid, chenodeoxycholic acid, zymosterol, diosgenin, fucosterol, fecosterol or a salt or ester thereof.

### Hydrophilic Polymer-Lipid Conjugate

In one embodiment, the lipid nanoparticle comprises a hydrophilic-polymer lipid conjugate capable of incorporation into the LNP. The conjugate includes a lipid covalently attached (optionally via a linker group) to a polymer chain that is hydrophilic. Examples of hydrophilic polymers include polyethyleneglycol (PEG), polyvinylpyrrolidone, polyvinylmethylether, polyhydroxypropyl methacrylate, polyhydroxypropylmethacrylamide, polyhydroxyethyl acrylate, polymethacrylamide, polydimethylacrylamide, polymethyloxazoline, polyethyloxazoline, polyhydroxyethyloxazoline, polyhydroxypropyloxazoline, polysarcosine and polyaspartamide. In one embodiment, the hydrophilic-polymer lipid conjugate is a PEG-lipid conjugate. The hydrophilic polymer lipid conjugate may also be a naturally-occurring or synthesized oligosaccharide-containing molecule, such as monosialoganglioside (GM1). The ability of a given hydrophilic-polymer lipid conjugate to enhance the circulation longevity of the LNPs herein could be readily determined by those of skill in the art using known methodologies. A PEG-lipid conjugate may be 1,2-dimyristoyl-rac-glycero-3-methoxypolyethylene glycol (DMG-PEG).

### Routes of Administration

In some embodiments, the lipid nanoparticle comprising mRNA is part of a pharmaceutical composition. The pharmaceutical composition may provide a prophylactic (preventative), ameliorative or a therapeutic benefit. The pharmaceutical composition will be administered at any suitable dosage. The type of administration used to introduce the LNP-mRNA includes any route that delivers the LNP-mRNA to a central nervous system (CNS) site in which the extracellular space comprises the interstitial and/or cerebrospinal fluid cerebrospinal fluid. The site includes the brain and/or spinal cord. The administration methods include but are not limited to intracerebroventricular (ICV) injection, lumbar intrathecal injection, cisterna magna injection and the use of a catheter (e.g., intrathecal catheter) to introduce the LNP-mRNA to the brain or spinal cord (see FIG. 10).

LNP-mRNA compositions as described herein may be administered to a subject. As used herein, a “subject” may be a human, non-human primate, rat, mouse, cow, horse, pig, sheep, goat, dog, cat, etc. The subject may be suspected of having or at risk for having a central nervous system disease, disorder, trauma or injury, are known to those of ordinary skill in the art. Some examples are listed in TABLE 1.

### Methods & Materials

### ICV Microinjections

All experimental protocols were approved by The University of British Columbia Committee on Animal Care and conducted in compliance with guidelines provided by the Canadian Council of Animal Care. Adult C57Bl6 mice (P40-P60) were anesthetized under 1-2% isoflurane and positioned on a stereotaxic frame. A small hole (diameter 1 mm) was drilled in the skull to allow access to the brain (−0.5 mm anterior/posterior (AP) and ±1.0 mm medial/lateral (ML) from bregma and −1.8 mm dorsal/ventral (DV)—see FIG. 10). A glass micropipette (tip diameter 40 μm) was connected to a Hamilton™ syringe and LNP-mRNAs were injected using an infusion pump (Harvard Apparatus™ Holliston, MA) at a rate of 200 nl/minute. The total volume injected was 1 μL of LNP-mRNA (0.05-1 mg mRNA/ml in sterile PBS). LNP-mRNA preparations were injected sequentially in the lateral ventricle of both hemispheres. After needle retraction, the skin on the skull was sutured, and mice were then single-housed. For mCherry™ imaging experiments, mice were kept for two days before transcardial perfusion with PBS and 4% paraformaldehyde for brain collection. For FGF-21 measurements in cerebrospinal fluid (CSF), mice were kept for 3 or 48 hours after injection, and CSF was then collected via a cisterna magna puncture (see FIG. 10).

IT Injections All experimental protocols were approved by The University of British Columbia Committee on Animal Care and conducted in compliance with guidelines provided by the Canadian Council of Animal Care. Adult C57Bl6 mice (P40-P60) mice were anesthetized under 1-2% isoflourane and fur around the lower spine was removed using an electric shaver. The mouse was positioned over a 15 mL conical tube under the abdomen and with a nose cone for continuous 1-2% isoflourane administration. LNP-mRNAs (5 μL) was injected using a Hamilton syringe (10 μL) between L5-L6.

### LNP Formulations

The LNPs were prepared by dissolving mRNA in 25 mM sodium acetate, pH 4.0, while the lipid components at the mole % specified were dissolved in absolute ethanol. The lipids in ethanol and the mRNA in buffer were combined in a 1:3 volume by volume ratio using a t-junction with dual-syringe pumps. The solutions were pushed through the t-junction at a combined flow rate of 20 mL/min (5 mL/minute for the lipid-containing syringe, 15 mL/minute for the mRNA-containing syringe). The mixture was subsequently dialyzed overnight against at least ˜100 volumes of 1× phosphate buffered saline, pH 7.4 using Spectro/Por dialysis membranes (molecular weight cut-off 12 000-14 000 Da). The LNPs were concentrated as required with an Amicon Ultra™ 10 000 MWCO (molecular weight cut-off), regenerated cellulose concentrator.

Lipid nanoparticles with the different formulations set forth in TABLE 2 below were assayed. The LNPs are composed of 4 lipid components: (1) an ionizable lipid (for example, nor-MC3 and Compound 22); (2) a non-cationic helper lipid (for example, DSPC or DOPG); (3) a sterol (for example, cholesterol); and (4) a polymer lipid conjugate (for example, PEG-DMG), as set out in TABLE 2 below. The LNPs were prepared with mRNA coding for fluorescent reporter protein mCherry™ or Fibroblast Growth factor 21 (FGF21) NM_020013.4 or Erythropoietin (EPO) NM_000799 loaded into LNPs. Fluorescent lipid dye DiO was included in some LNP formulations to allow tracking of LNP diffusion in the brain independent of reporter protein expression. For some experiments (LNPs A, B, C, and D), we used the lipophilic dye DiO (DiOC₁₈(3)), which is a green fluorescent, lipophilic carbocyanine dye that was incorporated into the LNP. The nitrogen-to-phosphate charge ratio was 6.

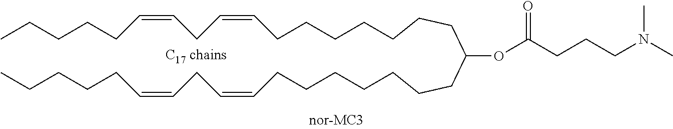 [SMILES: CCCCC/C=C\C/C=C\CCCCCCCC(CCCCCCC/C=C\C/C=C\CCCCC)OC(=O)CCCN(C)C]

TABLE 2
Lipid nanoparticle (LNP) formulations

| LNP | Cargo (mRNA) | LNP Components | Molar ratios (mol/mol) |
|---|---|---|---|
| A | mCherry | nMC3/DSPC/Chol/PEG-DMG/DiO | 50/10/39/0.5/0.5 |
| B | mCherry | nMC3/DSPC/Chol/PEG-DMG/DiO | 50/10/38/1.5/0.5 |
| C | mCherry | nMC3/DOPG/Chol/PEG-DMG/DiO | 50/10/39/0.5/0.5 |
| D | mCherry | nMC3/DOPG/Chol/PEG-DMG/DiO | 50/10/38/1.5/0.5 |
| E | mCherry | nMC3/DOPG/Chol/PEG-DMG | 50/10/38.5/1.5 |
| F | mCherry | compound 22/DOPG/Chol/PEG-DMG | 50/10/38.5/1.5 |
| G | FGF21 | nMC3/DOPG/Chol/PEG-DMG | 50/10/38.5/1.5 |
| H | FGF21 | compound 22/DOPG/Chol/PEG-DMG | 50/10/38.5/1.5 |
| I | EPO | compound 22/DOPG/Chol/PEG-DMG | 50/10/38.5/1.5 |

The ionizable, amino, cationic lipid nMC3 referred to in TABLE 2 above is the lipid referred to as nMC3 (nor-MC3) described at page 8 of WO 2022/246571 [2]. The ionizable, amino, cationic lipid, is compound 22 of PCT/CA2023/051274, titled “Amino Acid-Containing Ionizable Lipids for the Delivery of Therapeutic Agents”, having an international filing date of 27 Sep. 2023.

### Imaging

Brains were sliced in 300 μm-thick sections prior to immunostaining and imaging on a confocal microscope (Zeiss™). Individual cell types were labelled using common cell markers (rabbit anti-Olig2 1:200 for oligodendrocytes; rat anti-GFAP 1:500 for astrocytes and ependymal cells). Goat anti-rabbit (or anti-rat) secondary antibodies conjugated to Alexa-647™ dye were imaged using the 622 nm laser line. The lipophilic dye DiO (incorporated into the LNP) was imaged using the 488 nm laser line. The mCherry™ fluorescent protein was imaged using the 561 nm laser line.

### CSF Collection

CSF was collected as previously described [8]. Briefly, glass capillaries were pulled and trimmed so the inner diameter is approx. 0.5 mm. Mice were anaesthetized using the 3-Component anesthetic (Fentanyl™ 0.05 mg/kg, Midazolam 5 mg/kg, Dexmedetomidine 0.5 mg/kg). Eye ointment was applied and fur was shaved from the animal's neck and skull. Mice were fixed on a stereotaxic frame to form angle approximately 135° from the body, and a sagittal incision was made inferior to the occiput. Subcutaneous tissue was separated to expose the dura mater of the cisterna magna (see FIG. 10), and the dura mater was pierced with the capillary tube to draw CSF (2-10 μL). The CSF collected in the capillary was flushed into a clean tube and frozen immediately at −80° C. for future analysis.

### FGF-21 and EPO Measurements

CSF was collected from mice 3- or 48-hours after LNP-mRNA(FGF-21) or LNP-mRNA(EPO) injection. The presence of FGF-21 or EPO was quantified using a U-PLEX FGF-21 or U-PLEX EPO assay (Meso Scale Discovery™, USA).

### EXAMPLES

### Example 1: LNP-mRNA Leads to Broad Expression of a Reporter Protein in the Brain and Spinal Cord

To test for the feasibility of using brain cells to induce brain-wide diffusion of secretory proteins, the inventors first investigated the distribution pattern of LNP-mRNA and downstream protein expression following intracerebroventricular (ICV) injections. LNPs containing the lipophilic dye DiO and carrying mRNA encoding for mCherry™ were injected bilaterally in the lateral ventricles of adult (p40-60) mice. After 2 days to allow for the mCherry™ protein expression and accumulation, brains were collected and imaged.

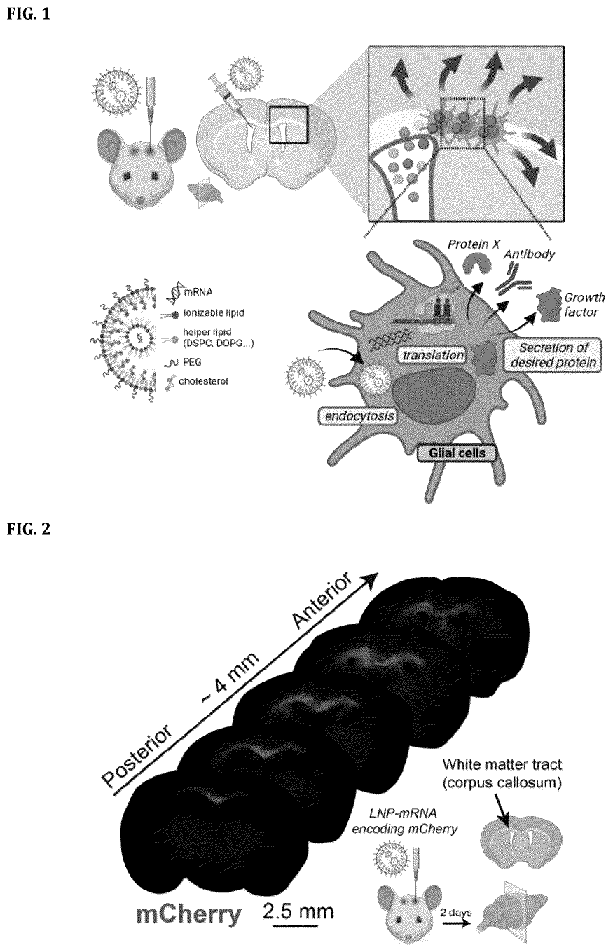

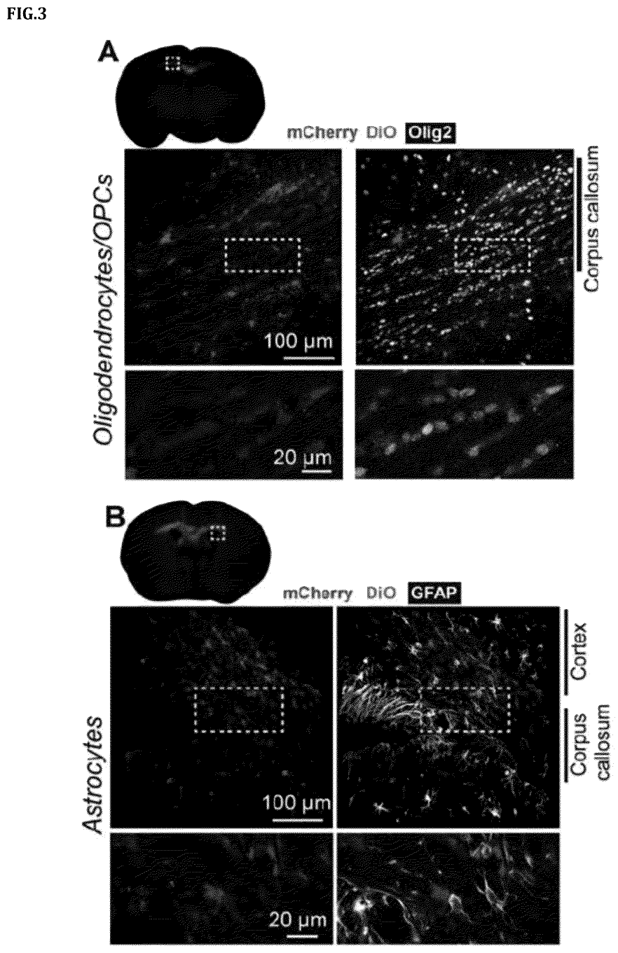

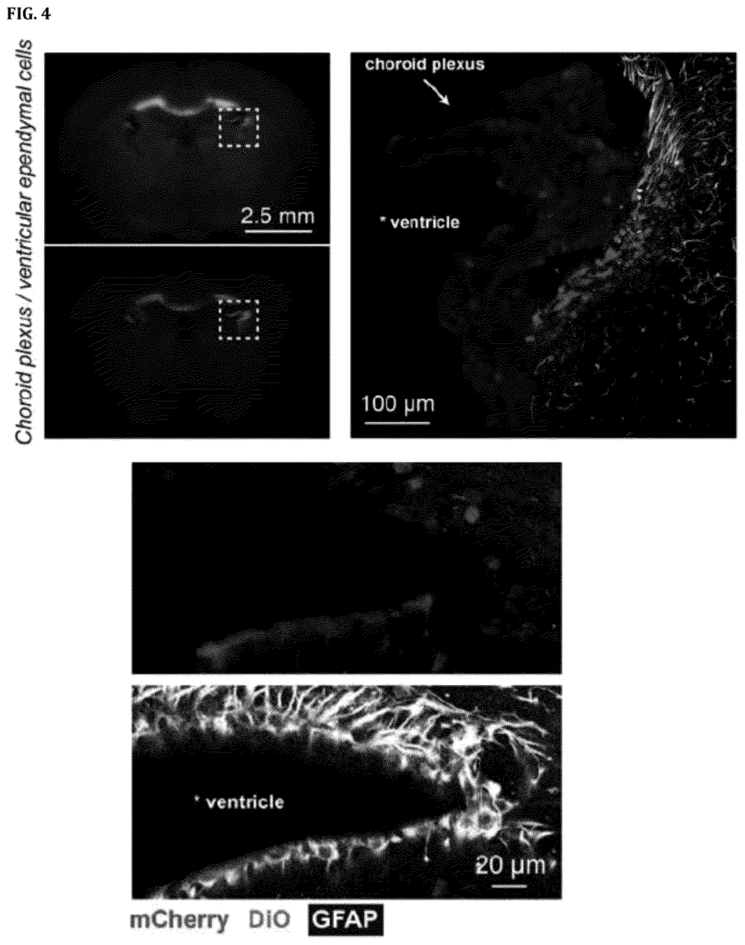

The distribution pattern of the DiO dye contained in the LNP formulation showed that LNPs had spread throughout the corpus collosum, a major white matter tract in the brain. Importantly, robust expression of the mCherry™ fluorescent protein reporter was readily observed along the corpus callosum, as well as along the ventricular wall and the choroid plexus within the ventricles. The reporter protein was expressed over a spread of >3 mm in the anterior-posterior axis, and >4 mm laterally (FIG. 2). Closer examination of the cell types producing mCherry™ within the white matter revealed expression in two glial cell types: oligodendrocytes, Olig2+ cells responsible for myelinating neuronal axons, as well as in astrocytes, immunostained for GFAP (FIG. 3). Furthermore, ependymal cells lining the ventricle wall as well as choroid plexus ependymal cells were found to express mCherry™ (FIG. 4). This shows that LNP-mRNA injection can be used to achieve extensive production of exogenous proteins within cells of the CNS.

In a separate experiment LNP-mRNA encoding for mCherry™ was injected intrathecally in the lumbar region between L5-L6. Robust expression of mCherry™ was observed along the entire length of the spinal cord (FIG. 5). This shows that LNP-mRNA injection can be used to achieve extensive production of exogenous proteins within cells of the CNS via multiple delivery routes.

### Example 2: LNP-mRNA Expression of a Secretory Protein Leads to Widespread Diffusion in the Brain

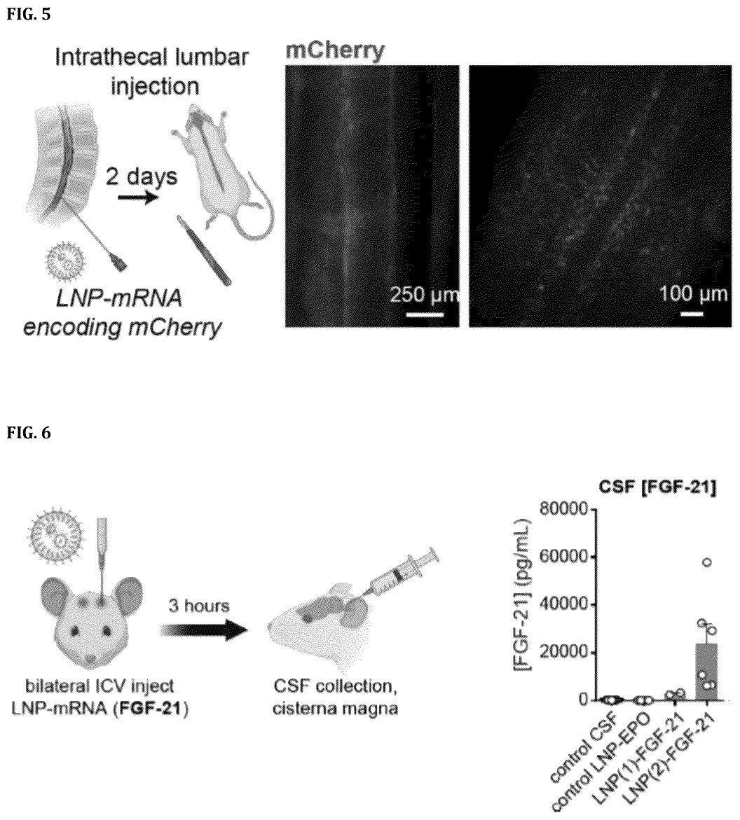

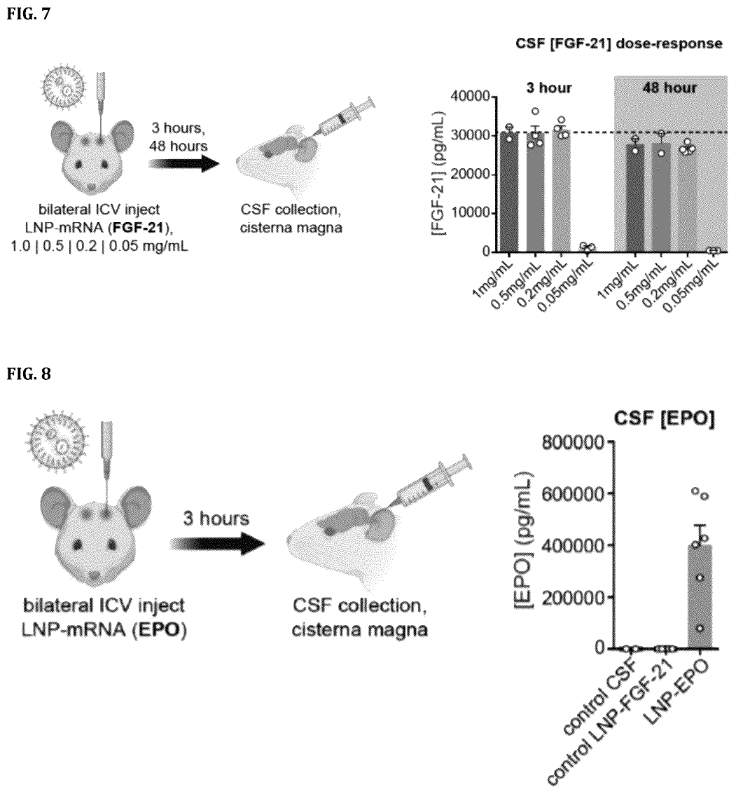

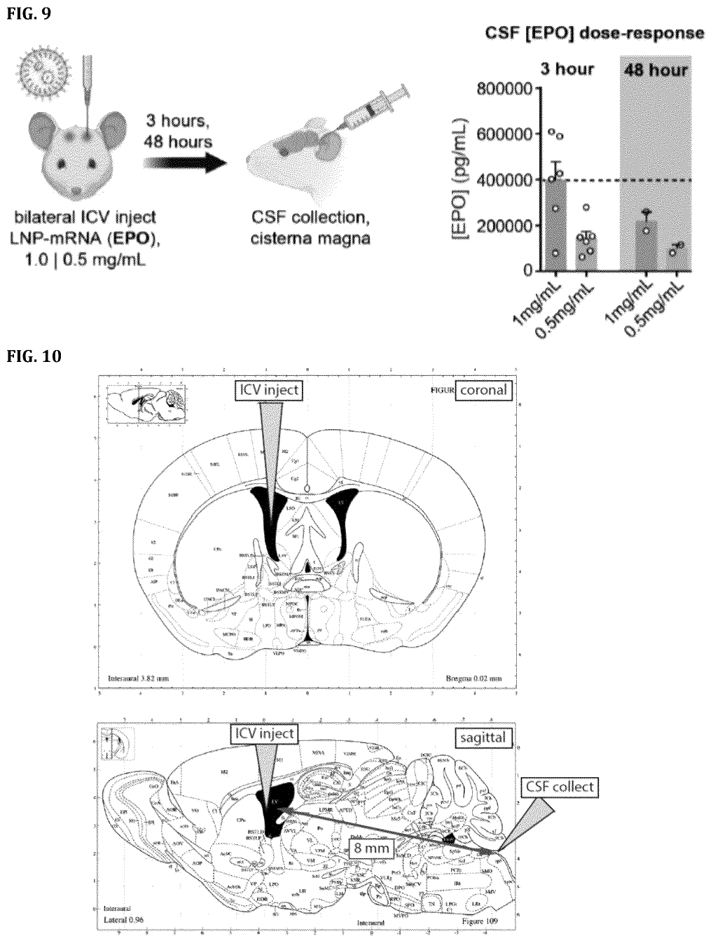

The broad distribution across white matter tracts as well as in structures responsible for CSF production suggests LNP-mRNA could be harnessed for the production of secretory proteins that would then diffuse across the CNS. The inventors tested this ‘bioreactor’ approach by injecting LNP(1)[or G]-mRNA-FGF-21 and LNP(2)[or H]-mRNA-FGF-21 as set out in TABLE 2 (obtained from NanoVation Therapeutics Inc.™) of LNP-mRNA encoding for Fibroblast growth factor 21 (FGF-21), a secreted growth factor that is primarily produced by hepatocytes and therefore normally virtually absent in the brain. Either three or 48 hours after bilateral intracerebroventricular injection in the brain, CSF was collected from the cisterna magna (the largest CSF-filled cistern in the brain) and quantified for the presence of FGF-21 using a U-PLEX assay. CSF FGF-21 concentrations were significantly higher for LNP(1)[or G]-mRNA-FGF-21 (2,790.3 pg/mL) and LNP(2)[or H]-mRNA-FGF-21 (23,765 pg/mL) over un-injected-(Control: 164.3 pg/mL) and injected controls (LNP-mRNA-EPO: 28.6 pg/mL) (FIG. 6). The levels of FGF21 in the CSF were elevated for a period of at least 48 hours (FIG. 7; LNP-mRNA-FGF-21 CSF collect 3 hr post-injection: 30,740 pg/mL; 48 hr: 27,719 pg/mL). Additionally, the levels of FGF-21 in the CSF varied with the LNP-mRNA concentrations injected, in a dose-dependent manner (FIG. 7; LNP-mRNA-FGF-21 1 mg/mL dose: 30,740 pg/mL; 0.5 mg/mL dose: 30,576 pg/mL; 0.2 mg/mL dose: 31,604 pg/mL; 0.05 mg/mL dose: 1,213 pg/mL). In a separate experiment, LNP-mRNA encoding for Erythropoietin (EPO) was injected intracerebroventricular. Either three or 48 hours after bilateral ventricular injection in the brain, CSF was collected from the cisterna magna and quantified for the presence of EPO using a U-PLEX assay. EPO was found in the CSF at high levels (FIG. 8; un-injected Control: 8.5 pg/ml; injection control LNP-FGF-21: 15.0 pg/ml; LNP-mRNA-EPO: 396,899 pg/ml). The levels of EPO in the CSF were elevated for a period of at least 48 hours (FIG. 9; LNP-mRNA-EPO 3 hr: 396,899 pg/mL; 48 hr: 144,286 pg/mL). Additionally, the levels of EPO in the CSF varied with the LNP-mRNA concentrations injected, in a dose-dependent manner (FIG. 9; LNP-mRNA-EPO 1 mg/mL dose: 396,899 pg/mL; 0.5 mg/mL dose: 218,629 pg/mL). The data shows that secretory proteins, such as growth factors or erythropoietin, can diffuse across the entire brain and reach site of actions that are distal to the actual site of LNP-mRNA uptake and expression. Surprisingly, the data shows that protein production, secretion and diffusion across the entire brain is consistent over a period of at least 48 hours.

### Example 3: In Vitro Method to Assess Whether a Protein Encoded by the mRNA is Capable of Secretion from a Cell of the Central Nervous System into Cerebrospinal Fluid

An in vitro primary culture assay is used to test the ability of cells of the central nervous system (e.g., astrocytes and oligodendrocytes (OL)) to secrete proteins from an LNP-mRNA. In parallel experiments, primary astrocytes and primary OL are cultured using common methods from perinatal mixed glial cultures (commonly known as the McCarthy and deVelis method). Briefly, brains from P0-P3 mice are collected, cortices are dissected and cells are mechanically dissociated through trituration. After successive washing steps, cells are plated and cultured for 3-6 days at 37° C., 5-8% CO₂. This gives rise to a “mixed glial culture”, composed of a bottom layer of astrocytes, on top of which is found a layer of oligodendrocyte progenitor cells (OPCs) as well as loosely adherent microglia. Shaking the plate induces detachment of microglia, leaving astrocytes and OPC. Further differential shaking steps then lead to OPC detachment. These OPCs are subsequently cultured separately and differentiated into mature OL using OL-specific culture media. In parallel, the remaining layer of astrocytes is passaged several times to obtain astrocyte-rich cultures. The purity of astrocyte and OL cultures can be confirmed using GFAP and O4/MBP immunostaining, respectively.

Astrocytes and OL cultures are used to assay the secretion of proteins. Primary cells (astrocytes or OL) are treated with LNP-mRNA coding for the protein. Secretion of the protein will be assayed by collecting the cell supernatant and quantifying the protein amount, via immunoblotting, ELISA or U-PLEX assay. The supernatant will be collected at various time points to estimate the peak production/secretion timeline. While the objective of the assay is focused on whether the protein is successfully secreted, it may also serve as a quantifiable assay for optimal LNP-mRNA formulation and concentration and to test the production/secretion efficiency of various mRNA constructs.

The detailed description and examples are intended to illustrate the preparation of specific lipid nanoparticle mRNA preparations and properties thereof but are in no way intended to limit the scope of the invention.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings.

### REFERENCES

(1) AKHTAR, A. et al. Neurodegenerative diseases and effective drug delivery: A review of challenges and novel therapeutics—J. Control Release (2021) 10:330:1152-1167.
(2) CIUFOLINI, M. A. et al. WO 2022/246571.
(3) DIMITROV, D. S. Therapeutic Proteins—Methods Mol Biol. (2012) 899: 1-26.
(4) FEIGIN, V. L. et al. The global burden of neurological disorders: translating evidence into policy—Lancet Neurol. (2020) 19(3): 255-265.
(5) GAL, L. et al. Restoration of Motor Function through Delayed Intraspinal Delivery of Human IL-10-Encoding Nucleoside-Modified mRNA after Spinal Cord Injury—Research (2023) 6:0056.
(6) GRIBKOFF, V. K. and KACZMAREK, L. K. Neuropharmacology. The Need for New Approaches in CNS Drug Discovery: Why Drugs Have Failed, and What Can Be Done to Improve Outcomes—(2017) 120: 11-19.
(7) LIEBNER, S. et al. Functional morphology of the blood-brain barrier in health and disease—Acta Neuropathol. 2018 March; 135(3): 311-336.
(8) LIU, L and DUFF, K. A Technique for Serial Collection of Cerebrospinal Fluid from the Cisterna Magna in Mouse—J Vis Exp. (2008) 21:960.
(9) NABHAN, J. F. et al. Intrathecal delivery of frataxin mRNA encapsulated in lipid nanoparticles to dorsal root ganglia as a potential therapeutic for Friedreich's ataxia—Sci Rep. (2016) 17:6:20019.
(10) PARDI, N. et al. WO 2023/086830.
(11) PARDRIDGE The Blood-Brain Barrier: Bottleneck in Brain Drug Development—NeuroRx. (2005) 2(1): 3-14.
(12) RUNGTA, R. L. et al. Lipid Nanoparticle Delivery of siRNA to Silence Neuronal Gene Expression in the Brain—Molecular Therapy—Nucleic Acids (2013) 2, e136.
(13) RUNGTA, R. L. et al. The Cellular Mechanisms of Neuronal Swelling Underlying Cytotoxic Edema—Cell (2015) 161:610-621.
(14) TANAKA, H. et al. In Vivo Introduction of mRNA Encapsulated in Lipid Nanoparticles to Brain Neuronal Cells and Astrocytes via Intracerebroventricular Administration—Mol. Pharmaceutics (2018) 15, 2060-2067.

1. A method for treating, preventing or diagnosing a central nervous system disease, disorder, trauma or injury, the method comprising:
contacting a lipid nanoparticle (LNP) with a cell of the central nervous system of a subject, the lipid nanoparticle comprising:
(a) an ionizable, amino, cationic lipid having a pKa between 5.0 and 7.0;
(b) a non-cationic helper lipid;
(c) a sterol;
(d) a hydrophilic polymer-lipid conjugate; and
(e) an mRNA encoding for a secretory polypeptide for treating, preventing or diagnosing the central nervous system disease, disorder, trauma or injury, the secretory polypeptide being capable of secretion from a cell of the central nervous system into a interstitial and/or cerebrospinal fluid of the subject,
wherein the contacting of the LNP with the cell results in the mRNA entering the cell and being translated in the cell, thereby resulting in production of the polypeptide,
wherein, subsequent to the production of the polypeptide, the polypeptide is secreted from the cell to a interstitial and/or cerebrospinal fluid (CSF) of the subject,
wherein the secreted polypeptide is present in the CSF of the subject at a first concentration at a first time point and at a second concentration at a later second time point,
wherein the first time point is 3 hours after contacting the LNP with the cell and the second time point is 48 hours after contacting of the LNP with the cell,
wherein the second concentration is at least 50%, at least 60%, at least 70%, at least 80%, at least 90%, or at least 95% of the first concentration, and
wherein the contacting is in vitro or in vivo.

2. The method of claim 1, wherein the cell of the central nervous system is selected from one or more of:
(a) a glial cell; and
(b) an ependymal cell.

3. The method of claim 2, wherein the glial cell is selected from an astrocyte or oligodendrocyte and the ependymal cell is from the cerebral ventricles or the choroid plexus.

4. The method of any one of claims 1-3, wherein the secretory polypeptide is endogenous to the cell of the central nervous system or modified to enhance its activity in the brain.

5. The method of any one of claims 1-4, wherein the secretory polypeptide is diffusible within the interstitial and/or cerebrospinal fluid of the subject.

6. The method of any one of claims 1-5, wherein the non-cationic helper lipid is a generally cylindrically-shaped lipid.

7. The method of any one of claims 1-6, wherein the non-cationic helper lipid is distearoylphosphatidylcholine (DSPC) or dioleoylphosphatidylglycerol (DOPG).

8. The method of any one of claims 1-7, wherein the non-cationic helper lipid has a phosphatidylethanolamine content that is less than 2 mol %.

9. The method of any one of claims 1-8, wherein the sterol is cholesterol.

10. The method of any one of claims 1-9, wherein the polymer lipid conjugate is PEG-DMG.

11. The method of any one of claims 1-10, wherein the molar ratio of ionizable, amino, cationic lipid/non-cationic helper lipid/sterol/polymer lipid conjugate is 50/10/38-39/1-2.

12. The method of any one of claims 1-11, wherein the administering comprises injection via an intracerebroventricular (ICV) or lumbar intrathecal route, cisterna magna route or via a catheter to the central nervous system of the subject.

13. Use of a pharmaceutical formulation comprising a lipid nanoparticle (LNP) for contacting a cell of the central nervous system of a subject to treat, prevent or diagnose a central nervous system disease, disorder, trauma or injury, the lipid nanoparticle comprising:
(a) an ionizable, amino, cationic lipid having a pKa between 5.0 and 7.0;
(b) a non-cationic helper lipid;
(c) a sterol;
(d) a hydrophilic polymer-lipid conjugate; and
(e) an mRNA encoding for a secretory polypeptide to treat, prevent or diagnose the central nervous system disease, disorder, trauma or injury, the secretory polypeptide being capable of secretion from a cell of the central nervous system into a interstitial and/or cerebrospinal fluid of a subject after administration of the lipid nanoparticle,
wherein the contacting of the LNP with the cell results in the mRNA entering the cell and being translated in the cell, thereby resulting in production of the polypeptide,
wherein, subsequent to the production of the polypeptide, the polypeptide is secreted from the cell to a interstitial and/or cerebrospinal fluid (CSF) of the subject,
wherein the secreted polypeptide is present in the CSF of the subject at a first concentration at a first time point and at a second concentration at a later second time point,
wherein the first time point is 3 hours after contacting the LNP with the cell and the second time point is 48 hours after contacting of the LNP with the cell,
wherein the second concentration is at least 50%, at least 60%, at least 70%, at least 80%, at least 90%, or at least 95% of the first concentration, and
wherein the contacting is in vitro or in vivo.

14. Use of a lipid nanoparticle for contacting a cell of the central nervous system of a subject for the manufacture of a medicament to treat, prevent or diagnose a central nervous system disease, disorder, trauma or injury, the lipid nanoparticle comprising:
(a) an ionizable, amino, cationic lipid having a pKa between 5.0 and 7.0;
(b) a non-cationic helper lipid;
(c) a sterol;
(d) a hydrophilic polymer-lipid conjugate; and
(e) an mRNA having a nucleic acid sequence encoding for a secretory polypeptide to treat, prevent or diagnose the central nervous system disease, disorder, trauma or injury, the secretory polypeptide being capable of secretion from a cell of the central nervous system into a interstitial and/or cerebrospinal fluid of a subject after administration of the lipid nanoparticle,
wherein the contacting of the LNP with the cell results in the mRNA entering the cell and being translated in the cell, thereby resulting in production of the polypeptide,
wherein, subsequent to the production of the polypeptide, the polypeptide is secreted from the cell to a interstitial and/or cerebrospinal fluid (CSF) of the subject,
wherein the secreted polypeptide is present in the CSF of the subject at a first concentration at a first time point and at a second concentration at a later second time point,
wherein the first time point is 3 hours after contacting the LNP with the cell and the second time point is 48 hours after contacting of the LNP with the cell,
wherein the second concentration is at least 50%, at least 60%, at least 70%, at least 80%, at least 90%, or at least 95% of the first concentration, and
wherein the contacting is in vitro or in vivo.

15. The use of claim 13 or 14, wherein the cell of the central nervous system is a glial cell selected from an astrocyte or oligodendrocyte or an ependymal cell of the cerebral ventricles or the choroid plexus.

16. The use of any one of claims 13-15, wherein the non-cationic lipid is a generally cylindrically-shaped lipid.

17. The use of any one of claims 13-16, wherein the non-cationic lipid is distearoylphosphatidylcholine (DSPC) or dioleoylphosphatidylglycerol (DOPG).

18. The use of any one of claims 13-17, wherein the phosphatidylethanolamine content is less than 2 mol %.

19. The use of any one of claims 13-18, wherein the sterol is cholesterol.

20. The use of any one of claims 13-19, wherein the polymer lipid conjugate is PEG-DMG.

21. The use of any one of claims 13-20, wherein the molar ratio of ionizable, amino, cationic lipid/non-cationic helper lipid/sterol/polymer lipid conjugate is 50/10/38-39/1-2.

22. The use of any one of claims 13-21, wherein the therapeutic polypeptide or peptide is endogenous to the cell of the central nervous system or modified to enhance its activity in the brain.

23. The use of any one of claims 13-22, wherein the therapeutic polypeptide or peptide is diffusible within the interstitial and/or cerebrospinal fluid.

24. A lipid nanoparticle for contacting a cell of the central nervous system of a subject to treat, prevent or diagnose a central nervous system disease, disorder, trauma or injury, the lipid nanoparticle comprising:
(a) an ionizable, amino, cationic lipid having a pKa between 5.0 and 7.0;
(b) a non-cationic helper lipid;
(c) a sterol;
(d) a hydrophilic polymer-lipid conjugate; and
(e) an mRNA having a nucleic acid sequence encoding for a secretory polypeptide for treating, preventing or diagnosing the central nervous system disease, disorder, trauma or injury, the secretory polypeptide being capable of secretion from a cell of the central nervous system into a interstitial and/or cerebrospinal fluid of a subject,
wherein the contacting of the LNP with the cell results in the mRNA entering the cell and being translated in the cell, thereby resulting in production of the polypeptide,
wherein, subsequent to the production of the polypeptide, the polypeptide is secreted from the cell to a interstitial and/or cerebrospinal fluid (CSF) of the subject,
wherein the secreted polypeptide is present in the CSF of the subject at a first concentration at a first time point and at a second concentration at a later second time point,
wherein the first time point is 3 hours after contacting the LNP with the cell and the second time point is 48 hours after contacting of the LNP with the cell,
wherein the second concentration is at least 50%, at least 60%, at least 70%, at least 80%, at least 90%, or at least 95% of the first concentration, and
wherein the contacting is in vitro or in vivo.

25. The lipid nanoparticle of claim 24, wherein the cell of the central nervous system is a glial cell selected from an astrocyte or oligodendrocyte or an ependymal cell of the cerebral ventricles or the choroid plexus.

26. The lipid nanoparticle of claim 24 or 25, wherein the lipid nanoparticle is part of a pharmaceutical formulation.

27. The lipid nanoparticle of any one of claims 24-26, wherein the secretory polypeptide is endogenous to the cell of the central nervous system or modified to enhance its activity in the brain.

28. The lipid nanoparticle of any one of claims 24-27, wherein the secretory polypeptide is diffusible within the interstitial and/or cerebrospinal fluid of the subject.

29. The lipid nanoparticle of any one of claims 24-28, wherein the non-cationic helper lipid is a generally cylindrical-shaped lipid.

30. The lipid nanoparticle of any one of claims 26-31, wherein the non-cationic lipid is distearoylphosphatidylcholine (DSPC) or dioleoylphosphatidylglycerol (DOPG).

31. The lipid nanoparticle of any one of claims 26-32, wherein the non-cationic helper lipid has a phosphatidylethanolamine content that is less than 2 mol %.

32. The lipid nanoparticle of any one of claims 24-31, wherein the sterol is cholesterol.

33. The lipid nanoparticle of any one of claims 24-32, wherein the polymer lipid conjugate is PEG-DMG.

34. The lipid nanoparticle of any one of claims 24-33, wherein the molar ratio of ionizable, amino, cationic lipid/non-cationic helper lipid/sterol/polymer lipid conjugate is 50/10/38-39/1-2.
